(19) United States
(12) Patent Application Publication
(10) Pub. No.: US 2022/0056397 A1
(43) Pub. Date: Feb. 24, 2022

(54) CELL ANALYSIS SYSTEMS

(71) Applicant: Hewlett-Packard Development Company, L.P., Spring, TX (US)

(72) Inventors: Alexander Govyadinov, Corvallis, OR (US); Viktor Shkolnikov, Palo Alto, CA (US)

(73) Assignee: Hewlett-Packard Development Company, L.P., Spring, TX (US)

(21) Appl. No.: 17/414,051

(22) PCT Filed: Feb. 1, 2019
(86) PCT No.: PCT/US2019/016332

§ 371 Date: Jun. 15, 2021

(51) Int. Cl. C12M 1/34; G01N 33/483; B01L 3/00; C12M 1/00; C12M 3/06; C12M 1/36; C12N 1/06

(52) U.S. Cl. CPC C12M 41/42; G01N 33/4833; B01L 3/502715; C12M 29/18; B01L 2300/023; C12M 41/48; C12N 1/066; B01L 2300/0663; C12M 27/16

(57) ABSTRACT

In one example in accordance with the present disclosure, a cell analysis system is described. The cell analysis system includes at least one cell analysis device. Each cell analysis device includes a channel to serially feed individual cells from a volume of cells into a lysing chamber. The cell analysis device also includes at least one feedback-controlled lysing element in the lysing chamber to agitate a cell. The cell analysis system also includes a controller to analyze the cell. The controller includes a lysate analyzer to analyze properties of the lysate and a rupture analyzer to analyze parameters of an agitation when a cell membrane ruptures.

### BACKGROUND

Analytic chemistry is a field of chemistry that uses instruments to separate, identify, and quantify matter. Cell lysis is a process of rupturing the cell membrane to extract intracellular components for purposes such as purifying the components, retrieving deoxyribonucleic acid (DNA), ribonucleic acid (RNA), proteins, polypeptides, metabolites, or other small molecules contained therein, and analyzing the components for genetic and/or disease characteristics. Cell lysis bursts a cell membrane and frees the inner components. The fluid resulting from the bursting of the cell is referred to as lysate.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a cell analysis system, according to an example of the principles described herein.

FIG. 2 is a flow chart of a method of cell analysis based on lysate properties and agitation parameters, according to an example of the principles described herein.

FIG. 3 is an operational diagram of a cell analysis system, according to an example of the principles described herein.

FIG. 4 is a side view of a cell analysis system, according to an example of the principles described herein.

FIG. 5 is a view of a cell analysis device of a cell analysis system, according to another example of the principles described herein.

FIG. 6 is a view of a cell analysis device of a cell analysis system, according to another example of the principles described herein.

FIG. 7 is a side view of a cell analysis system, according to another example of the principles described herein.

FIG. 8 is a view of a cell analysis device of a cell analysis system, according to another example of the principles described herein.

FIG. 9 is a view of a cell analysis device of a cell analysis system, according to another example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Cellular analytics is a field of chemistry that uses instruments to separate, identify, and quantify matter. A wealth of information can be collected from a cellular sample. For example, the mechanical properties of the cell membrane and even more specifically information relating to the mechanical breakdown of the cell membrane can provide insight to the characteristics and state of a cellular sample. For example, in some cases the physical characteristics of a particular cell can be used to classify and/or differentiate the particular cell from other cells. In another example, changes to the physical characteristics of a cell can be used to determine a state of the cell. For example, parasitic invasion of a cell—such as occurs in cells affected by malaria—can alter the membrane of the cell. Gross changes to tissue, such as when cancer is present in a cell, can also alter the physical properties of the cell membrane. In other words, cell membrane strength indicates cell membrane composition and cell composition. Accordingly, a cell analysis system that can measure cell membrane strength provides to an individual, information regarding the cell membrane composition from which characteristics of the cell can be determined.

The intracellular components of the cell also provide valuable information about a cell. Cell lysis is a process of extracting intracellular components from a cell and can also provide valuable information about a cell. During lysis, the intracellular components are extracted for purposes such as purifying the components, retrieving DNA and RNA proteins, polypeptides, metabolites, and small molecules or other components therein, and analyzing the components for genetic and/or disease characteristics. Cell lysis ruptures a cell membrane and frees the inner components. The fluid containing the inner components is referred to as lysate. The contents of the cell can then be analyzed by a downstream system.

The study and analysis of the lysate of a cell provides information used to characterize and analyze a cell. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

While cellular analytics is useful in cellular analysis, refinements to the operation may yield more detailed analysis results. For example, in general it may be difficult to obtain a correlation between 1) the mechanical and chemical properties of a cell and 2) the genetic information of the cell. Knowing this correlation can have many advantages. For example, a correlation between genomic information and a cells susceptibility to lysis may allow a prediction of lytic antibiotic resistance of a cell based on the cells genetic information. Accordingly, both pieces of information, i.e., mechanical properties and genetic information, for a cell are valuable and useful in analytic chemistry.

In general, it is difficult to acquire both pieces of information from a particular sample and a scientist may have to pick from between the two pieces of information (e.g., mechanical and genomic), which they would like to collect. It may be more desirable to obtain the genomic information from the cell as it provides more information. However as described above, the mechanical properties of a cell also provide valuable information. For example, lysis information allows a user to infer cell mechanical properties which may indicate to the user the state of the cell, i.e., dead/living, diseased/healthy.

Accordingly a user cannot simultaneously get mechanical and genetic information from a single sample. To get both genomic and mechanical information, two different samples would be used. However, as the different samples may have different properties, any correlation between the separately collected genomic and mechanical information would rely on a similarity between the two samples, which similarity may not exist or may be tenuous. Accordingly, it may be desirable to obtain genomic and mechanical properties from a single sample so as to remove inter-sample variation from any resulting correlation.

This simultaneous measurement of genomic and mechanical properties of a cell is advantageous in a variety of circumstances. Disease pathology is a specific example as mechanical properties play a particular role in disease pathology. For example, the elasticity (mechanical property) of a circulating tumor cell may be a determining factor of the cell's metastatic potential and therefore may be an indicator of cancerous cells. In this example, the genetic information collected form a sample indicates what mutations are activated in the cell and may indicate which pathways are up or down regulated. From the genetic and mechanical information, a medical professional may determine which chemotherapy to prescribe as the role of many chemotherapeutics is to affect these pathways. As yet another example, malaria, which is a parasitic infection of red blood cells that changes a stiffness (mechanical property) of the red blood cells and changes the transportation of these cells through the circulatory system. By obtaining the genetic information at the same time, a scientist may determine a type of parasite (there are many malarial parasites for example) that are affecting the patient. With such detailed solutions, a more specific anti-malarial process may be followed.

While some solutions have been presented, they are inadequate for any number of reasons. For example, flow cytometry is an example of a single cell analysis technique. Flow cytometry differentiates cells based on their spatial scattering profile or their bulk fluorescence. However, flow cytometry does not obtain genetic and/or mechanical information about a cell. To obtain the mechanical properties of a cell, deformation flow cytometry may be performed which combines differentiation based on fluorescence and scattering with cell deformation behavior. However, deformation flow cytometry does not obtain genetic information.

In some cases, single cell genomic analysis may be performed. In this example, cell solutions are diluted and aliquoted into wells. The wells are lysed indiscriminately and after certain preparation operations, the genetic material is sequenced. However, this does not indicate any mechanical information about the cell nor of the cell membrane.

Accordingly, the present specification describes a system for simultaneously obtaining genetic (RNA, DNA) information and mechanical information of a cell population with single cell resolution in an automated fashion on a large number of cells. The system includes a reservoir that holds a cell suspension, a flow structure that segregates the cells such that they enter single file into a lysing chamber. Within the lysing chamber a feedback-controlled lysing operation is carried out. Information regarding the lysing operation and the properties of the lysate are passed to a controller to analyze the cell based on both pieces of information. Accordingly, valuable information from precious cell populations can be made even when the number of cells to be analyzed cannot be increased.

Specifically, the present specification describes a cell analysis system that includes at least one cell analysis device. Each cell analysis device includes a channel to serially feed individual cells from a volume of cells into a lysing chamber. Each cell analysis device also includes at least one feedback-controlled lysing element in the lysing chamber to agitate the cell. A controller of the cell analysis system analyzes the cell and includes 1) a lysate analyzer to analyze properties of the lysate and 2) a rupture analyzer to analyze parameters of the agitation when a cell membrane ruptures.

In another example, the cell analysis system includes a cell reservoir to hold a volume of cells to be analyzed. In this example, the cell analysis system includes a microfluidic cell analysis die with a substrate. At least one cell analysis device is formed in the substrate. In this example, each cell analysis device includes a channel to serially feed individual cells from a volume of cells into a lysing chamber. At least one feedback-controlled lysing element is in the lysing chamber to agitate the cell. An ejector, responsive to a determination that the cell membrane has ruptured, ejects the lysate. The system also includes a controller to analyze the cell. The controller includes a lysate analyzer to analyze properties of the lysate and a rupture analyzer to analyze parameters of the agitation when a cell membrane ruptures. Moreover, in this example, the system includes a pump to move the cells through the at least one cell analysis device and a waste reservoir to collect waste fluid.

The present specification also describes a method. According to the method, a quantity of cells is passed from a cell reservoir to at least one cell analysis device of an underlying microfluidic cell analysis die. For each cell analysis device, a feedback-controlled lysing element is activated in a lysing chamber of the cell analysis device. The feedback-controlled lysing element is to agitate the cell. Responsive to a determination that the cell membrane has ruptured, lysate information is passed to a lysate analyzer and parameters of the agitation when a cell membrane ruptures are passed to a rupture analyzer. Based on the output of both the rupture analyzer and the lysate analyzer, the cell is analyzed.

In summary, using such a cell analytic system 1) allows single cell analysis of a sample; 2) allows combined cell analysis, i.e., a genetic analysis and a mechanical property analysis; 3) can be integrated onto a lab-on-a-chip; 4) is scalable and can be parallelized for high throughput, and 5) is low cost and effective. However, the devices disclosed herein may address other matters and deficiencies in a number of technical areas.

As used in the present specification and in the appended claims, the term “cell membrane” refers to any enclosing structure of a cell, organelle, or other cellular particle.

Further, as used in the present specification and in the appended claims, the term “agitation cycle” refers to a period when a cell is exposed to the operations of a lysing element. For example, an agitation cycle may refer to each time a cell is looped past a single lysing element. In another example, a cell passes through an agitation cycle each time it passes by a lysing element in a string of multiple lysing elements.

Even further, as used in the present specification and in the appended claims, the term “rupture threshold” refers to the amount of stress that a cell can withstand before rupturing. In other words, the rupture threshold is the threshold at which the cell ruptures. The rupture threshold may be determined based on any number of factors including a number of agitation cycles a cell is exposed to and the intensity of the agitation cycles.

Yet further, as used in the present specification and in the appended claims, the term “parameters” refers to the operating conditions in a particular agitation cycle. For example, a “parameter” may refer to a type of lysing element and/or a lysing strength. For example, agitation parameters for an agitation cycle may include whether a lysing element is a thermal inkjet resistor, a piezo-electric device, or an ultrasonic transducer. Agitation parameters also refer to the operating conditions of the particular lysing element. For example, the parameters of an ultrasonic transducer may refer to the frequency, amplitude, and/or phase of ultrasonic waves. The parameters of the thermal inkjet resistor and piezo-electric device may refer to the size of the element and/or the voltage applied to the element.

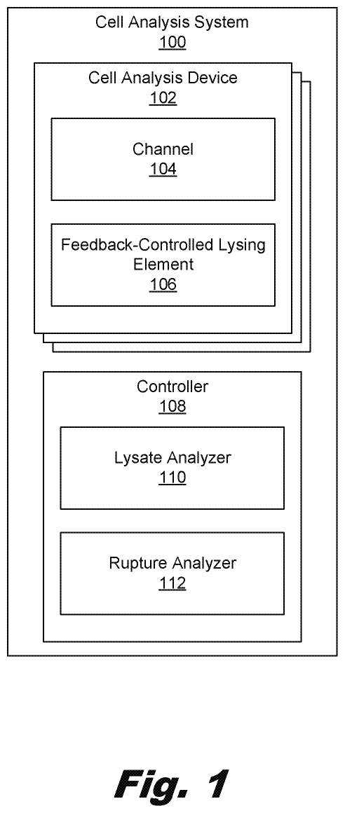

Turning now to the figures, FIG. 1 is a block diagram of a cell analysis system (100), according to an example of the principles described herein. In some examples, the cell analysis system (100) is part of a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small scale fluid analysis functionality.

In other words, the components, i.e., the cell analysis device(s) (102), channel(s) (104), and feedback-controlled lysing element(s) (106) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The cell analysis system (100) includes at least one cell analysis device (102) which performs the cellular analysis. In some examples, a single cell analysis device (102) is used in the cell analysis system (100). However, the cell analysis system (100) may include multiple cell analysis devices (102), each to analyze an individual cell. In this example, the multiple cell analysis devices (102) may be in parallel. The multiple parallel cell analysis devices (102) facilitate the processing of more cells. For example, as described above, each cell analysis device (102) analyzes a single cell. Accordingly, multiple parallel cell analysis devices (102) allow multiple cells to be analyzed at the same time, rather than analyzing a single cell at a time.

To carry out such analysis, each cell analysis device (102) includes a variety of sub-components. Specifically, the cell analysis device(s) (102) includes a channel (104) to direct incoming cells into a lysing chamber. Accordingly, the channel (104) is coupled at one end to a cell reservoir and directs cells single-file into a lysing chamber.

The lysing chamber is a location where lysing and lysis detection occur. In some examples the lysing chamber may be no more than 100 times a volume of a cell to be lysed. In other examples, the lysing chamber may have a cross-sectional size comparable with the cell size and in some cases smaller than the cell so as to deform the cell before or during the rupturing of the cell membrane. That is, the lysing chamber may be a microfluidic structure.

As the lysing chamber is the location where lysis occurs, the lysing chamber receives a cell or other component to be lysed. In some examples, the lysing chamber may receive the cells single-file, or serially. Thus, lysing operations can be performed on a single cell and that cell's particular properties may be analyzed and processed.

In general, lysis refers to the agitation of a cell with the objective of rupturing a cell membrane. Lysis ruptures a cellular particle membrane and frees the inner components. The fluid containing the inner components is referred to as lysate. The contents of the cellular particle can then be analyzed by a downstream system.

Accordingly, the lysing chamber includes a feedback-controlled lysing element (106) to carry out such an agitation. The feedback-controlled lysing element (106) may implement any number of agitation mechanisms, including shearing, ball milling, pestle grinding, and using rotating blades to grind the membranes. Other examples of agitation mechanisms include localized heating and shearing by constriction. In another example, repeated cycles of freezing and thawing can disrupt cells through ice crystal formation. Solution-based lysis is yet another example. In these examples, the osmotic pressure in the cellular particle could be increased or decreased to collapse the cell membrane or to cause the membrane to burst. As yet another example, the cells may be forced through a narrow space, thereby shearing the cell membranes.

In one example, the feedback-controlled lysing element (106) is a thermal inkjet heating resistor disposed within the lysing chamber. In this example, the thermal inkjet resistor heats up in response to an applied current. As the resistor heats up, a portion of the fluid in the chamber vaporizes to generate a bubble. This bubble generates a pressure and shear spike which ruptures the cell membrane.

In another example, the feedback-controlled lysing element (106) may be a piezoelectric device. As a voltage is applied, the piezoelectric device changes shape which generates a pressure pulse in the chamber that generates a pressure and shear spike which ruptures the cell membrane.

In yet another example, the feedback-controlled lysing element (106) may be a non-reversible electroporation electrode that forms nano-scale pores on the cell membrane. These pores grow and envelope the entire cell membrane leading to membrane lysis. In yet another example, the feedback-controlled lysing element (106) is an ultrasonic transducer that generates high energy sonic waves. These high energy waves may travel through the wall of the chamber to shear the cells disposed therein.

The different types of feedback-controlled lysing elements (106) each may exhibit a different agitation mechanism. For example, the agitation mechanism of an ultrasonic transducer is the ultrasonic waves that are emitted and that shear the cells. The agitation mechanism of the thermal inkjet heating resistor is the vapor bubble that is generated and ruptures the cell membrane. The agitation mechanism of the piezo-electric device is the pressure wave that is generated during deformation of the piezo-electric device, which pressure wave shears the cell membrane. While particular examples of feedback-controlled lysing elements (106) have been described herein, a variety of feedback-controlled lysing element (106) types may be implemented in accordance with the principles described herein.

A feedback-controlled lysing element (106) refers to a lysing element (106) whose operation is monitored to ensure lysis occurs as desired. That is, the feedback-controlled lysing element (106) provides a quality control check over a lysing operation. In this example, the lysing chamber includes a sensor to determine when a cell has ruptured, and to return the cell to within range of the feedback-controlled lysing element (106) in the case the cell has not ruptured. That is, the sensor detects a change in the cell based on an agitation of the cell by the at least one feedback-controlled lysing element (106). If no change is detected, the cell is kept in, or returned to, the lysing chamber for another agitation cycle. Accordingly, rather than activating the feedback-controlled lysing element (106) and hoping that lysing occurs, a feedback-controlled lysing element (106) includes a sensor to ensure lysing occurs prior to further processing of the lysate.

The cell analysis system (100) also includes a controller (108) that analyzes the cells of the sample. The controller (108) includes various components to make such an analysis. First, the controller (108) includes a lysate analyzer (110) to receive information regarding the lysate. That is, after the cell has been ruptured, the contents therein may be analyzed and information provided to the lysate analyzer (110). A variety of pieces of information can be collected from the lysate. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

The controller also includes a rupture analyzer (112) which determines a rupture threshold of the cell based on the parameters of the agitation when the cell membrane ruptures. That is, as described above a cell may be exposed to one or multiple agitation cycles. The parameters of the different agitation cycles can be passed to the rupture analyzer (112) which determines a rupture threshold. The rupture analyzer (112) may use this information to perform a variety of analytical operations. For example, the rupture analyzer (112) may differentiate cells in a sample based on different rupture thresholds. In this example, the rupture analyzer (112) may receive, for multiple cells, information regarding the results of lysing by different feedback-controlled lysing element(s) (106) on those cells. Based on the results, the rupture analyzer (112) may determine when each cell in a sample is ruptured. Different types of cells may rupture under different intensities. Accordingly, based on when a cell ruptures, the rupture analyzer (112) may be able to determine the cell types of the various cells in a sample.

As another example, the rupture analyzer (112) may be able to determine a state of a cellular sample. For example, it may be determined that healthy cells rupture at a particular lysing intensity. This may be determined by passing healthy cells through the cell analysis system (100) and collecting rupturing information. Accordingly, a sample to be analyzed may subsequently be passed through the cell analysis system (100) and rupturing information collected for these cells in the sample. If the rupturing information indicates that the sample cells rupture at a lower intensity than the healthy cells, the rupture analyzer (112) may determine that the sample cells are diseased.

As yet another example, the rupture analyzer (112) may be able to differentiate between live cells and dead cells based on the rupturing thresholds of different cells as determined by the cell analysis device (102). That is, live cells may be more robust against lysing and therefore have a higher rupturing threshold as compared to dead cells which may rupture at a lower intensity.

Thus, the present cell analysis system (100) provides a way to collect information related to both the lysate and the mechanical properties of the cell membrane from a single sample. Being able to collect both pieces from a single sample removes any bias resulting from intra-sample variation. For example, both the elasticity of a circulating tumor cell as well as the genetic components of the tumor cell may be determined from a single sample. As yet another example, both a stiffness of a red blood cell as well as the genetic aspects of the cell can be analyzed to determine if the cell is affected by malaria. Being able to collect both pieces of information from a single sample also makes more effective use of the sample. That is, rather than requiring two groups of the sample, one for mechanical testing and one for genetic testing, both pieces of information from one group of the sample.

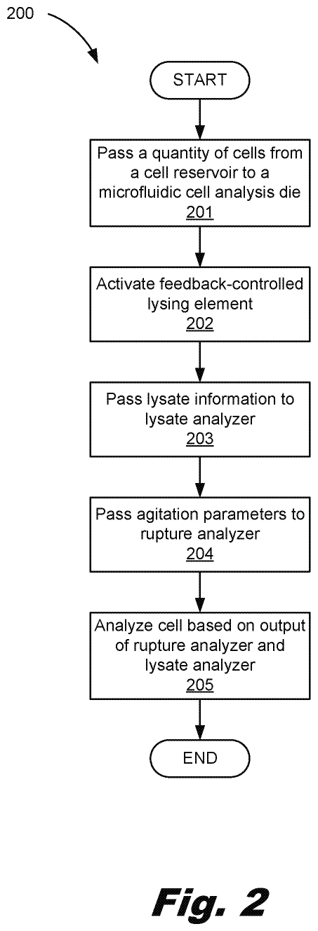

FIG. 2 is a flow chart of a method (200) of cell analysis based on lysate properties and agitation parameters, according to an example of the principles described herein. In the method (200), a quantity of cells to be analyzed are passed (block 201) from a cell reservoir to a cell analysis die. As described above, the cell analysis die and the components thereof may be microfluidic structures. As used in the present specification and in the appended claims, the term cell analysis die refers to a substrate with multiple microfluidic cell analysis devices (FIG. 1, 102) disposed thereon. The substrate may be formed of any material including plastic and silicon, such as in a printed circuit board. The cell reservoir may be any structure that holds a quantity of cells to be analyzed.

In some examples, the quantity of cells are serially passed (block 201) to each cell analysis device (FIG. 1, 102) of the microfluidic cell analysis die. That is, each cell within the sample may be received (block 201) one at a time. In some examples, each cell analysis device (FIG. 1, 102) includes a channel (FIG. 1, 104) that gates introduction of one cell at a time into the lysing chamber for agitation. Such a serial, single-file introduction of cells into each cell analysis device (FIG. 1, 102) may be facilitated by channels (FIG. 1, 104) having a cross-sectional area size on the order of the cell diameter. Such single-file, or serial, inlet of cells facilitates an individual analysis of cells. Accordingly, rather than analyzing a portion of the sample and extrapolating therefrom, each cell of the sample may be analyzed. Thus, a complete analysis of the sample is performed. As described above, the cellular analysis die may include any number of cell analysis devices (FIG. 1, 102). Thus, while each cell analysis device (FIG. 1, 102) analyzes a single cell at a time, a die with multiple cell analysis devices (FIG. 1, 102) may analyze the cells in parallel. Doing so may increase throughout.

With a cell present in a corresponding lysing chamber, a feedback-controlled lysing element (FIG. 1, 106) is activated (block 202) for each cell analysis device (FIG. 1, 102). As described above, lysing is an operation wherein a cell is agitated until its membrane ruptures or otherwise breaks down. The point at which a cell membrane breaks down may be referred to as a rupture threshold and may provide valuable information about a particular cell as described above.

In some examples, the feedback-controlled lysing element (FIG. 1, 106) may be a feedback-controlled lysing element (FIG. 1, 106). That is, in some examples, a lysing element may not rupture a cell membrane. For example, the cell membrane may be robust against a particular intensity of agitation. Without feedback-controlled lysis, the cell may leave the lysing chamber intact. Outputting an intact cell when a lysed cell is desired and/or expected, may result in skewed results. Accordingly, when a sensor indicates that, despite the operations of the feedback-controlled lysing element (FIG. 1, 106), the cell membrane has not ruptured, the controller (FIG. 1, 108) may activate a return pump to return the cell to be under the influence of the feedback-controlled lysing element (FIG. 1, 106). In this example, a second lysing cycle, either at the same intensity or an increased intensity, may be executed such that the cell may be ruptured. That is in some examples, agitation intensity may be incrementally adjusted until the cell membrane ruptures.

That is, once in the cell analysis device (FIG. 1, 102), the cell is exposed to repeated agitation cycles. In this example, the lysing intensity may increase or remain the same. For example, the feedback-controlled lysing element (FIG. 1, 106) may be adjustable. As specific examples, the energy applied to a thermal inkjet resistor may increase or the intensity of ultrasonic waves may increase with each agitation cycle. In another example, the feedback-controlled lysing element (FIG. 1, 106) may have a fixed intensity. In either case, the cell is exposed to the agitation cycles until the cell ruptures. As described above, cell rupture may be determined by the sensor of the cell analysis device (FIG. 1, 102). That is, the sensor, whatever type it may be, can detect the difference between an intact cell and a ruptured cell. Accordingly, such a feedback-controlled lysing operation ensures that a cell that is intended to be lysed is in fact lysed.

The rupturing of the cell membrane triggers various actions. First, lysate information may be passed (block 203) to a lysate analyzer (FIG. 1, 110). For example, genetic information collected from a DNA cell may be passed to the lysate analyzer (FIG. 1, 110). Additionally, parameters of the agitation that resulted in the cell membrane rupture may be passed (block 204) to the rupture analyzer (FIG. 1, 112). That is, the controller (FIG. 1, 108) may record information relating to a type of agitation mechanism, a strength of the agitation cycles, and a number of cycles a cell was exposed to before ultimately rupturing. Put another way, the rupture analyzer (FIG. 1, 112) can determine the rupture threshold by knowing how many agitation cycles the cell was exposed to and the intensity of each agitation cycle. Thus, the rupture analyzer (FIG. 1, 112) determines at what agitation intensity the cell ultimately ruptures. With such information on hand, the rupture analyzer (FIG. 1, 112) can determine certain properties of the cell including cell type, cell state, etc.

Using both of these outputs, the cell is analyzed (block 205). That is, rather than just analyzing one characteristic, i.e., lysate information or mechanical property information, both can be analyzed on a single cell. The dual analysis of a cell thus provides more information than would be possible by targeting a single type of information. That is, while a first sample could be tested for mechanical properties and a second sample could be tested for genetic information, inherent differences between the samples may skew results. However, by analyzing both pieces of information from a single sample, an accurate and reliable mapping between mechanical properties, i.e., rupture threshold and lysate properties, may be made. Such a method (200) also promotes sample efficiency by collecting more information from a sample, thus fewer cells will be exhausted during a cell analysis operation.

In some examples, the above described method (200) is performed on genomic samples. For example, the quantity of cells may be genetic cells, the lysate is a nucleic acid, and the method is to sequence the genetic cells. In one particular example, a sample of genetic material may be introduced into the reservoir and individual genetic cells may be individually introduced into different cell analysis devices (FIG. 1, 102). Each may be agitated by the feedback-controlled lysing element (FIG. 1, 106) until rupture occurs. The parameters of the agitation that led to the genetic cell rupture are passed to the rupture analyzer (FIG. 1, 112) and the DNA and/or RNA lysed from the cells is passed to the lysate analyzer (FIG. 1, 110). The results of the analyzers may be combined to provide an overall analysis of the cell. For example, whether the cell is diseased may be determined based on an output of the rupture analyzer (FIG. 1, 112) and the genetic makeup of the cell determined from the lysate analyzer (FIG. 1, 110). Thus, an overall analysis of the cell may indicate a genetic disposition to suffer from a particular disease may be made. As a specific example, the sample may be mixed with polymerase chain reaction (PCR) primers and master mix and an amplification executed. If the amplification occurs, the sequences of interest are present in the sample. While particular reference is made to one type of sequencing, other types of genetic sequencing are also facilitated by the method (200) and cell analysis system (FIG. 1, 100).

Another specific example is now provided. In this example, a swab fluid, for example phosphate buffered saline wash from a swab of a counter in a food processing plant, containing bacteria (e.g., salmonella or listeria, may be introduced into the cell analysis system (FIG. 1, 100). In this example, a stain marks bacteria in the sample to determine if the bacteria is Gram negative or positive. Based on this analysis, the bacteria cells are sorted and concentrated. The bacteria is then introduced through the channel (FIG. 1, 104) into the lysing chamber where it is acted upon by the feedback-controlled lysing element (FIG. 1, 106). In this example, the operating parameters of the feedback-controlled lysing element (FIG. 1, 106), i.e., a strength of the applied energy is tuned based on whether the cell is Gram negative or Gram positive. The cell is exposed to multiple iterations of lysing to make sure the bacteria cells are lysed. The lysate which includes the disturbed cell membrane and cytoplasm: proteins, peptides, nucleic acid material, lipids, small molecules, sugars is encapsulated into a droplet and ejected to be further analyzed.

In this example, an encapsulated droplet is merged with a droplet of master mix containing primers of interest, and then diluted, and split into a large number (e.g. 10,000-100,000) droplets and then each is thermocycled. In a few of those droplets, amplification takes place and is detected. Such a process may be referred to as a digital droplet polymerase chain reaction (PCR). The droplets are counted and a quantity of the desired nucleic acid material in the bacteria may be ascertained.

Another specific example of the method (200) is now provided. In this example, a test is performed to identify bacteria in food manufacturing. In this example, Swab fluid containing bacteria is collected by swabbing surfaces of a food processing plant. This fluid is introduced into the cell analysis system (FIG. 1, 100) reservoir. One at a time these cells pass through channels (FIG. 1, 104) of each cell analysis device (FIG. 1, 102) and into the lysis chambers. Via the feedback-controlled lysing elements (FIG. 1, 106) and rupture analyzer (FIG. 1, 112), the strength needed to lyse these bacteria is determined. From this information, a scientist may infer the bacteria type. As an example, gram positive bacteria has a thick peptidoglycan layer and so it is tougher to lyse then gram negative bacteria. Based on this information primers are chosen for a PCR reaction with the lysate to type the bacteria further. However, even based on the gram +/− information, cell wall targeting antibiotics may be prescribed for cleaning or may be recommended for individuals that may have consumed food from the contaminated food processing plant.

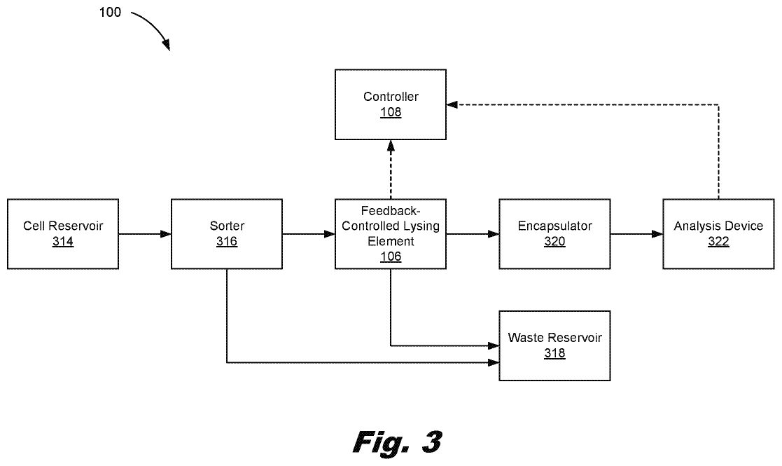

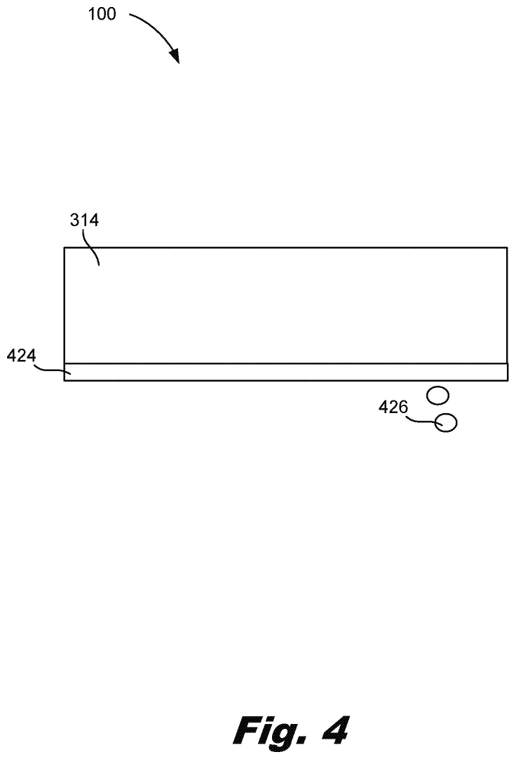

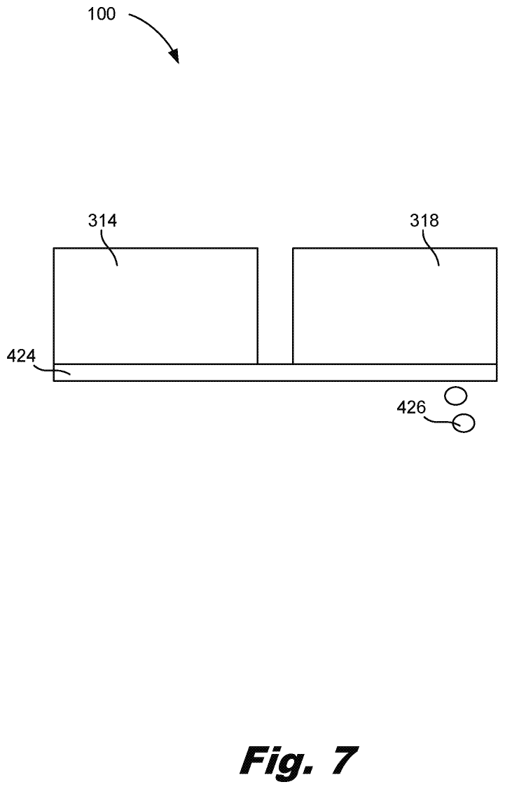

FIG. 3 is an operational diagram of a cell analysis system (100), according to an example of the principles described herein. That is, FIG. 3 depicts the path different components of a cellular sample take through the cell analysis system (100). First, as described above the cell sample may be retained in a cell reservoir (314), which may be any container or receptacle to hold a sample of cells to be analyzed by the cell analysis devices (FIG. 1, 102). The cell reservoir (314) may be coupled to each of multiple cell analysis devices (FIG. 1, 102). For example as depicted in FIGS. 4 and 7, the cell reservoir (314) may be disposed on top of the microfluidic cell analysis die which contains the cell analysis devices (FIG. 1, 102).

In this example, prior to passing to the lysing chamber where the cell is to be agitated, the cells in the sample may be passed through a sorter (316) which separates the cells to be analyzed from a carrier fluid. For example, a particular sample may include a variety of cells, but a single type of cell may be desired to be analyzed by the cell analysis system (100). Accordingly, the sorter (316) separates the desired cell to be analyzed from other cells in the sample and/or the carrier fluid of the sample. Doing so provides a more concentrated solution of the cells. Moreover, by excluding undesirable cell types from being analyzed, any results are more particularly mapped to the desired cell. That is, the results of an analysis of a particular cell would not be skewed by analysis of a disparate cell type.

In some examples, the disparate cells and/or carrier fluid is ejected to a waste reservoir (318). In other words, the waste reservoir (318) is coupled to each cell analysis device (FIG. 1, 102) and collects byproducts of the sorting. As will be described below, the waste reservoir (318) may also collect waste fluid resulting from the lysis operation.

The cell is then passed by the feedback-controlled lysing element (106) where it is exposed to agitation cycles until it ruptures. As described above, in some examples, the byproducts, or waste from this operation is also passed to the waste reservoir (318).

In some examples, each cell analysis device (FIG. 1, 102) includes an encapsulator (320) to encapsulate the lysate. That is, following lysis the lysates of various cells may intermingle one with another. The diffusing of the lysate of various cells may obfuscate analysis. Accordingly, the encapsulator (320) encapsulates each lysate with a casing to prevent mixture with other lysates, thus preserving it for subsequent, and individual analysis.

Following encapsulation, the lysate is passed to a downstream analysis device (322) for subsequent analysis. The downstream analysis device (322) may be of any type. For example, the downstream analysis device (322) may be perform a subsequent lysing operation to further break down the components of the cell. In yet another example, the downstream analysis device (322) may be a microarray, or a titration plate. As yet another example, the downstream analysis device (322) may perform PCR on the cell lysate.

In some examples, the downstream analysis device (322) may be a component of the cell analysis system (FIG. 1, 100) and/or device (FIG. 1, 102). That is, the downstream analysis device (322) may be formed in the same silicon substrate as the other components, albeit in a different chamber. In yet another example, the downstream analysis device (322) may be a separate component, for example a well plate to which the lysate is ejected.

In either case, information from the downstream analysis device (322) and from the feedback-controlled lysing element (106) is passed to a controller (108) for analysis and processing. That is, the controller (108) receives multiple types of information, 1) i.e., genomic/lysate information and 2) rupturing information from which a detailed cell analysis can be executed.

FIG. 4 is a side view of a cell analysis system (100), according to an example of the principles described herein. As described above, the cell analysis system (100) includes a cell reservoir (314) fluidically coupled upstream of each cell analysis device (FIG. 1, 102) to hold the volume of cells that are to be analyzed. FIG. 4 also depicts the microfluidic cell analysis die (424) where cell analysis occurs. That is, the microfluidic cell analysis die (424) includes multiple cell analysis devices (FIG. 1, 102). Cells are passed to each cell analysis device (FIG. 1, 102) in parallel even though each cell analysis device (FIG. 1, 102) may analyze a single cell at a time.

In the example depicted in FIG. 4, the microfluidic cell analysis die (424) is disposed under the cell reservoir (314). In this example, gravity, or a pressure draw from a pump, pulls the cells into the microfluidic cell analysis die (424). The cell sample is then passed through each cell analysis device (FIG. 1, 100). In some examples, as depicted in FIGS. 4-6, the waste reservoir (318) is disposed in the microfluidic cell analysis die (424) and more specifically as a component of the cell analysis device (FIG. 1, 102). That is, at least one of the waste reservoir (318) and a pump are formed in the microfluidic cell analysis die (424).

As described above, in some examples, the lysate (426) is ejected onto a surface. Accordingly, in this example, the microfluidic cell analysis die (424) or individual cell analysis devices (FIG. 1, 102) include ejectors to eject the lysate to the intended surface, i.e., the well plate.

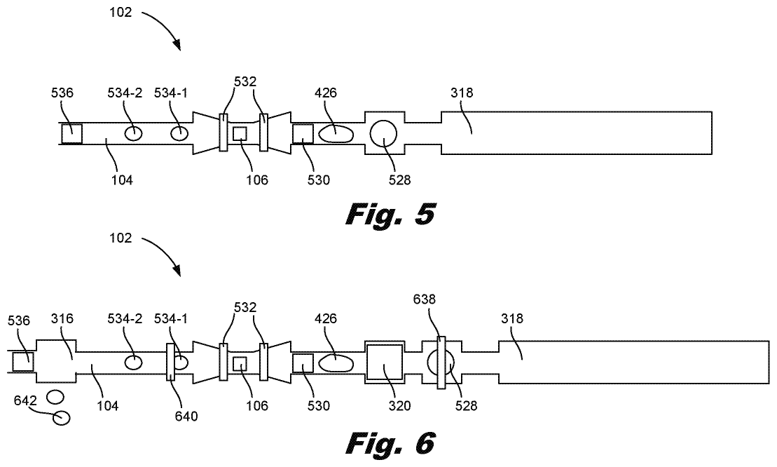

FIG. 5 is a view of a cell analysis device (102) of a cell analysis system (FIG. 1, 100), according to another example of the principles described herein. Specifically, FIG. 5 is an example of the cell analysis device (102) of a cell analysis system (FIG. 1, 100) depicted in FIG. 4 where the waste reservoir (318) is disposed in the microfluidic cell analysis die (424). That is, in some examples, the cell analysis system (100) also includes a waste reservoir fluidically coupled downstream of each cell analysis device (FIG. 1, 102) to collect waste fluid. As depicted in FIG. 5, the waste reservoir (318) may be formed in the silicon substrate that underlies the cell reservoir (FIG. 3, 314).

In this example, the cell analysis device (102) includes a pump (536) to move the cells (534-1, 534-2) through the cell analysis device (102). In some examples, the pump (536) may be an integrated pump, meaning the pump (536) is integrated into a wall of the channel (104). In some examples, the pump (536) may be an inertial pump which refers to a pump (536) which is in an asymmetric position within the channel (104). The asymmetric positioning within the channel (104) facilitates an asymmetric response of the fluid to the pump (536). The asymmetric response results in fluid displacement when the pump (536) is actuated. In some examples, the pump (536) may be a thermal inkjet resistor, or a piezo-drive membrane or any other displacement device. While FIG. 5 depicts a pump (536) per cell analysis device (102), the pump (536) may be disposed upstream of the cell analyses devices (102) for example in a manifold. In this example, the single pump (536) may direct fluid to all the cell analysis devices (102) found in a microfluidic cell analysis die (FIG. 4, 424).

FIG. 5 also depicts the cells (534-1, 534-2) as they pass through the channel (104) towards the feedback-controlled lysing element (106). As described above, the cells (534-1, 534-2) may be passed single-file down the channel (104) such that each is individually agitated and potentially ejected. Thus, rather than analyzing results extrapolated from the agitation of a few cells, the results represent the agitation of each cell in a sample.

FIG. 5 also depicts the feedback-controlled lysing element (106) and other components that facilitate the feedback-controlled lysing operation. As described above the feedback-controlled lysing element (106) may take many forms such as a thermal inkjet heating resistor, a piezoelectric device, a non-reversible electroporation electrode, an ultrasonic transducer, or another device that relies on another agitation mechanism such as shearing, ball milling, pestle grinding, using rotating blades to grind the membrane, localized heating, shearing by constriction, repeated cycles of freezing and thawing, and solution-based lysis among others. While particular reference is made to a few types of feedback-controlled lysing elements (106) any variety of feedback-controlled lysing elements (106) may be implemented in accordance with the principles described herein.

FIG. 5 also depicts the sensor (532) used to determine whether the cell membrane was ruptured and a return pump (530) to re-direct a cell (534) to the lysing chamber when the cell membrane has not ruptured. The sensor (532) may take many forms. For example, the sensor (532) may be an optical scatter sensor that determines cell rupture based on a scatter of reflected energy waves. The sensor (532) may be an optical fluorescence sensor that detects cell rupture based on the detection of certain fluorescent markers. In other examples, the sensor (532) may be an optical bright field sensing system, an optical dark field sensing system, or a thermal property sensor.

In one particular example, the sensor (532) is an impedance sensor. Specifically, the sensor (532) may include at least one pair of electrodes spaced apart from one another by a gap. These electrodes detect a level of conductivity within the gap. That is, incoming cells to a lysing chamber, and the solution in which they are contained, have a predetermined electrical conductivity. Any change to the contents within the lysing chamber will effectively change the electrical conductivity within the lysing chamber. Specifically, as the cells are ruptured and the nucleic acid pours out, the conductivity would increase. To measure the conductivity, a resistance of solution between electrodes of the impedance sensor is measured and a conductivity determined therefrom. In some examples, a single pair of electrodes are used, with one electrode plate placed at either end of a chamber. In another example, multiple pair of electrodes are used. For example, one pair of electrode plates could be placed at the inlet and another pair of electrode plates placed at the outlet.

Thus, in summary, the sensor (532) which may include one sensor (532) in the lysing chamber or which may include multiple sensors (532) in the lysing chamber, can determine when a cell (534) membrane has been ruptured.

The cell analysis device (102) also includes a return pump (530) to return an un-ruptured cell to be within the region of the single feedback-controlled lysing element (106) where the cell (534) may be again exposed to the operation of the feedback-controlled lysing element (106). This shifting of the un-ruptured cell to be by the feedback-controlled lysing element (106) may be continued until the cell (534) is ruptured and the lysate (426) passed down the system.

As described above, information regarding the parameters (type, strength, and count) of the agitation cycles are passed to a controller (FIG. 1, 108) which determines a rupture threshold of the cell (534) based on the parameters of the agitation when the cell (534) membrane ruptures. That is, as described above a cell (534) may be exposed to gradually increasing intensities of lysing operations. The characteristics of the different agitation cycles can be passed to the controller (FIG. 1, 108) which determines a rupture threshold.

In some examples, the cell analysis device (102) gradually increases the intensity of agitation such that it can be precisely determined at what stress level a particular cell (534) ruptures. Increasing the agitation intensity may include increasing the intensity of the feedback-controlled lysing element (106) and/or by increasing a count of how many exposures the cell (534) has to the feedback-controlled lysing element (106). For example, a feedback-controlled lysing element (106) intensity may not change, but the cell (534) may be passed by the feedback-controlled lysing element (106) multiple times until cell rupture occurs. In another example, a feedback-controlled lysing element (106) intensity increases and the cell may be passed by the feedback-controlled lysing element (106) multiple times until cell rupture occurs.

FIG. 5 also depicts the ejector (528) that expels the lysate (426) fluid. That is, each cell analysis device (FIG. 1, 102) includes an ejector (528) to eject the lysate (426). The lysate (426) may be expelled by the ejector (528) to a downstream analysis device (FIG. 3, 322) for further analysis while the waste fluid is expelled to a waste reservoir (318) for collection and disposal.

The ejector (528) may include a firing resistor or other thermal device, a piezoelectric element, or other mechanism for ejecting fluid from the firing chamber. For example, the ejector (528) may be a firing resistor. The firing resistor heats up in response to an applied voltage. As the firing resistor heats up, a portion of the fluid in the firing chamber vaporizes to form a bubble. This bubble pushes the lysate (426) out the opening and onto a surface such as a micro-well plate. As the vaporized fluid bubble collapses, a vacuum pressure along with capillary force within the firing chamber draws lysate (426) into the firing chamber from a reservoir, and the process repeats. In this example, the ejector (528) may be a thermal inkjet ejector (528).

In another example, the ejector (528) may be a piezoelectric device. As a voltage is applied, the piezoelectric device changes shape which generates a pressure pulse in the firing chamber that pushes a fluid out the opening and onto the print medium. In this example, the ejector (528) may be a piezoelectric inkjet ejector (528).

FIG. 6 is a view of a cell analysis device (102) of a cell analysis system (FIG. 1, 100), according to another example of the principles described herein. Specifically, FIG. 6 is an example of the cell analysis device (102) of the cell analysis system (FIG. 1, 100) depicted in FIG. 4 where the waste reservoir (318) is disposed in the microfluidic cell analysis die (FIG. 4, 424). That is, the waste reservoir (318) may be formed in the silicon substrate that underlies the cell reservoir (FIG. 3, 314). FIG. 6 depicts various components depicted in FIG. 5 including the pump (536), cells (534-1, 534-2), channel (104), feedback-controlled lysing element (106), sensor (532), return pump (530), lysate (426), ejector (528), and waste reservoir (318).

FIG. 6 also depicts a number of additional components. For example, FIG. 6 depicts the sorter (316) that operates to separate the cells (534) from waste fluid (642). As describe above, this waste fluid (642) may include carrier fluid and/or cells within a sample that are not intended to be analyzed. The waste fluid (642) may be routed to a different waste reservoir outside of the cell analysis system (FIG. 1, 100). In the example depicted in FIG. 6, the cell analysis device (102) includes a cell presence sensor (640) to detect the presence of a cell (534) to be lysed in the cell analysis device (FIG. 1, 102). The cell presence sensor (640) may activate the at least one feedback-controlled lysing element (106) based on a detected presence of the cell (534) to be lysed. The cell presence sensor (640) may be of any variety of types such as those described as examples of sensor (532). That is, the cell presence sensor (640) may be an impedance sensor, an optical scatter sensor, an optical fluorescence sensor, an optical bright field imaging system, an optical dark field imaging system, or a thermal property sensor. This cell presence sensor (640) is disposed before the feedback-controlled lysing element (106) and may trigger activation of the feedback-controlled lysing element (106). For example, if the cell presence sensor (640) sends information to the controller (FIG. 1, 108) which indicates that a cell (534) is not present, the controller (FIG. 1, 108) may avoid activating the feedback-controlled lysing element (106). By comparison, if the cell presence sensor (640) sends information to the controller (FIG. 1, 108) which indicates that a cell (534) is present, the controller (FIG. 1, 108) may activate the feedback-controlled lysing element (106). FIG. 6 also depicts the encapsulator (320), which as described above encapsulates a lysate (426).

In some examples, the cell analysis device (102) includes a lysate sensor (638) to trigger the action of the ejector (528). That is, the lysate sensor detects a presence of the lysate (426) and activates the ejector (528) based on a detected presence of the lysate. In this example, the lysate sensor (638) may be of any variety of types such as those described as examples of sensor (532). That is, the lysate sensor (638) may be an impedance sensor, an optical scatter sensor, an optical fluorescence sensor, an optical bright field imaging system, an optical dark field imaging system, or a thermal property sensor. This lysate sensor (638) is disposed in the firing chamber, or upstream of the firing chamber, where the ejector (528) is located and may trigger activation of the ejector (528). For example, if the lysate sensor (638) sends information to the controller (FIG. 1, 108) which indicates that a lysate (FIG. 4, 426) is not present within the firing chamber or that a non-lysate component is disposed within the firing chamber, the controller (FIG. 1, 108) may avoid activating the ejector (528). By comparison, if the lysate sensor (638) sends information to the controller (FIG. 1, 108) which indicates that a lysate (FIG. 4, 426) is present, the controller (FIG. 1, 108) may activate the ejector (528) to eject the lysate (FIG. 4, 426) from the system.

FIGS. 7-9 depict an example of the cell analysis device (102) where the waste reservoir (318) is not disposed in the microfluidic cell analysis die (424), but is disposed above the microfluidic cell analysis die (424). That is, in some examples, the microfluidic cell analysis die (424) is disposed under the cell reservoir (314) and the waste reservoir (318).

Specifically, FIG. 7 is a side view of a cell analysis system (100), according to an example of the principles described herein. As described above, the cell analysis system (100) includes a cell reservoir (314) fluidically coupled upstream of each cell analysis device (FIG. 1, 102) to hold the volume of cells that are to be analyzed. FIG. 7 also depicts the waste reservoir (318) fluidically coupled downstream of each cell analysis device (FIG. 1, 102) to collect waste fluid. In this example, the operation of the main pump (FIG. 5, 536), another pump, or fluid mechanics may draw fluid into the waste reservoir (318) from the microfluidic cell analysis die (424).

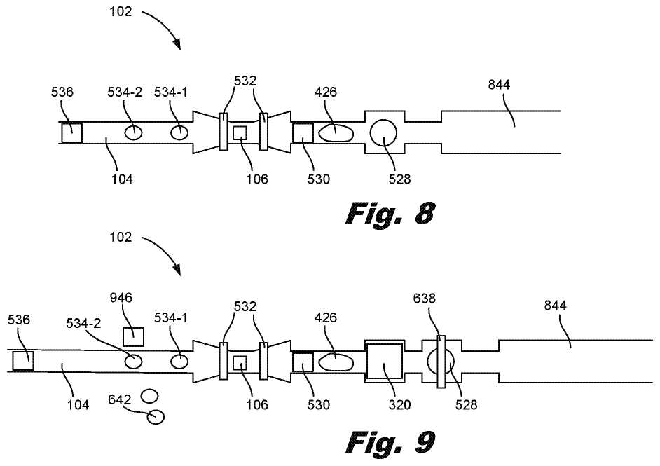

FIG. 8 is a view of a cell analysis device (102) of a cell analysis system (FIG. 1, 100), according to another example of the principles described herein. Specifically, FIG. 8 is an example of the cell analysis device (102) of the cell analysis system (FIG. 1, 100) depicted in FIG. 7 where the waste reservoir (FIG. 3, 318) is disposed on top of the microfluidic cell analysis die (FIG. 4, 424). In this example, rather than having the waste reservoir (FIG. 3, 318) disposed in the substrate of the microfluidic cell analysis die (FIG. 4, 424), the substrate includes a waste channel (844) that directs the fluid to the waste reservoir (FIG. 3, 318). FIG. 8 also depicts various components previously described such as, the pump (536), channel (104), cells (534), feedback-controlled lysing element (106), sensor (532), return pump (530), lysate (426), and ejector (528).

FIG. 9 is a view of a cell analysis device (FIG. 1, 102) of a cell analysis system (FIG. 1, 100), according to another example of the principles described herein. Specifically, FIG. 9 is an example of the cell analysis device (102) of the cell analysis system (FIG. 1, 100) depicted in FIG. 7 where the waste reservoir (FIG. 3, 318) is disposed on top of the microfluidic cell analysis die (FIG. 4, 424).

FIG. 9 depicts various components depicted in FIG. 8 including the pump (536), cells (534-1, 534-2), channel (104), feedback-controlled lysing element (106), sensor (532), return pump (530), lysate (426), ejector (528), and waste channel (844).

FIG. 9 also depicts a number of additional components. For example, FIG. 9 depicts a detector (946). The detector (946) detects a marker that is found on the cells to be analyzed. That is, the marker may be formed on cells (534) to be analyzed and the detector (946) senses these markers. For example, the detector (946) may be an example of a sorter (FIG. 3, 316) that operates to separate the cells (534) from waste fluid (642). That is, cells (534) with a marker detected by the detector (946) may be allowed to pass while cells (534) without a detected marker are ejected as waste fluid (642).

In another example, an output of the detector (946) selectively activates the feedback-controlled lysing element (106). That is, the detector (946) may be an example of a cell presence detector (FIG. 6, 640) that is upstream of the lysing chamber and triggers activation of the feedback-controlled lysing element (106). FIG. 9 also depicts the encapsulator (320) and lysate sensor (638) described above.

In summary, using such a cell analytic system 1) allows single cell analysis of a sample; 2) allows combined cell analysis, i.e., a genetic analysis and a mechanical property analysis; 3) can be integrated onto a lab-on-a-chip; 5) is scalable and can be parallelized for high throughput, and 6) is low cost and effective. However, the devices disclosed herein may address other matters and deficiencies in a number of technical areas.

What is claimed is:

1. A cell analysis system, comprising:
at least one cell analysis device, each cell analysis device comprising:
a channel to serially feed individual cells from a volume of cells into a lysing chamber;
at least one feedback-controlled lysing element in the lysing chamber to agitate a cell;
a controller to analyze the cell, the controller comprising:
a lysate analyzer to analyze properties of a lysate of the cell; and
a rupture analyzer to analyze parameters of an agitation when a cell membrane ruptures.

2. The cell analysis system of claim 1, wherein the lysing chamber further comprises:
a sensor to determine whether the cell membrane was ruptured; and
a return pump to re-direct a cell to the lysing chamber when the cell membrane has not ruptured.

3. The cell analysis system of claim 1, wherein each cell analysis device further comprises an encapsulator to encapsulate the lysate.

4. The cell analysis system of claim 1, further comprising:
a cell reservoir fluidically coupled upstream of each cell analysis device to hold the volume of cells; and
a waste reservoir fluidically coupled downstream of each cell analysis device to collect waste fluid.

5. The cell analysis system of claim 1, wherein each cell analysis device further comprises an ejector to eject the lysate.

6. The cell analysis system of claim 5, wherein each cell analysis device further comprises a lysate sensor to:
detect a presence of the lysate; and
activate the ejector based on a detected presence of the lysate.

7. The cell analysis system of claim 1, wherein each cell analysis device further comprises a cell presence sensor to:
detect a presence of a cell to be lysed in the cell analysis device; and
activate the at least one feedback-controlled lysing element based on a detected presence of the cell to be lysed.

8. A cell analysis system, comprising:
a cell reservoir to hold a volume of cells to be analyzed;
a microfluidic cell analysis die comprising:
at least one cell analysis device formed in a substrate, each cell analysis device comprising:
a channel to serially feed individual cells from a volume of cells into a lysing chamber;
at least one feedback-controlled lysing element in the lysing chamber to agitate a cell;
an ejector to, responsive to a determination that a cell membrane has ruptured, eject a lysate of the cell; and
a controller to analyze the cell, the controller comprising:
a lysate analyzer to analyze properties of the lysate; and
a rupture analyzer to analyze parameters of an agitation when a cell membrane ruptures;
a pump to move the cells through the at least one cell analysis device; and
a waste reservoir to collect waste fluid.

9. The cell analysis system of claim 8, wherein the microfluidic cell analysis die is disposed under the cell reservoir and the waste reservoir.

10. The cell analysis system of claim 8, wherein at least one of the waste reservoir and the pump are formed in the microfluidic cell analysis die.

11. The cell analysis system of claim 8, further comprising a sorter to separate the cells to be analyzed from a carrier fluid.

12. The cell analysis system of claim 8, further comprising a detector of a marker of the cells to be analyzed, wherein:
the marker is formed on the cells to be analyzed; and
an output of the detector selectively activates the feedback-controlled lysing element.

13. A method, comprising:
passing, a quantity of cells from a cell reservoir to at least one cell analysis device of an underlying microfluidic cell analysis die;
for each cell analysis device, activating a feedback-controlled lysing element in a lysing chamber of the cell analysis device, wherein the feedback-controlled lysing element is to agitate the cell;
responsive to a determination that a cell membrane has ruptured:
passing lysate information to a lysate analyzer; and
passing parameters of an agitation when a cell membrane ruptures to a rupture analyzer; and
analyzing the cell based on output of both the rupture analyzer and the lysate analyzer.

14. The method of claim 13, further comprising incrementally adjusting agitation intensity until the cell membrane ruptures.

15. The method of claim 13, wherein:
the quantity of cells are genetic cells;
the lysate is a nucleic acid; and
the method is to sequence the genetic cells.
